(19) United States
(12) Patent Application Publication
(10) Pub. No.: US 2007/0134177 A1
(43) Pub. Date: Jun. 14, 2007

(54) Effect Materials

(76) Inventor: Curtis J. Zimmermann, Cold Spring, NY (US)

Correspondence Address: BASF CATALYSTS LLC, 100 CAMPUS DRIVE, FLORHAM PARK, NJ, 07932, US

(21) Appl. No.: 11/538,484

(22) Filed: Oct. 4, 2006

Related U.S. Application Data
(60) Provisional application No. 60/724,356, filed on Oct. 6, 2005.

(51) Int. Cl. A61K 8/29; A61K 8/27

(52) U.S. Cl. 424/61; 424/401; 424/63

(57) ABSTRACT

The present invention provides a cosmetic comprising pearlescent pigment. The pearlescent pigment comprises: (a) a CVD or PVD deposited layer of a first material; and (b) a CVD or PVD deposited layer of a second material.

This patent application claims priority to pending provisional patent application 60/724,356 filed Oct. 6, 2005 incorporated herein in its entirety by reference.

### BACKGROUND OF THE INVENTION

The present invention relates to effect materials.

The use of effect pigments, also known as pearlescent pigments or nacreous pigments, in order to impart a pearlescent luster, metallic luster and/or multi-color effect approaching iridescent, is well-known. Effect pigments are composed of a plurality of laminar substrates, each of which is coated with one or more reflecting/transmitting layers. Pigments of this type were first based on metal oxides, as described in U.S. Pat. Nos. 3,087,828 and 3,087,829, and a description of their properties can be found in L. M. Greenstein, “Nacreous (Pearlescent) Pigments and Interference Pigments”. Pigment Handbook, Volume I, Properties and Economics, Second Edition, pp. 829-858, John Wiley & Sons, NY 1988.

The unique appearance of effect pigments is the result of multiple reflections and transmissions of light. The substrate usually has a refractive index which is different from the coating and usually also has a degree of transparency. The coating is in the form of one or more thin layers which have been deposited on the substrate. If more than one layer is used, the layers are made of materials with different refractive indices. Pearlescent luster is derived from specular reflection from the surfaces that are essentially parallel to each other.

One important aspect of the coating on the substrate is that it must be smooth and uniform in order to achieve the optimum pearlescent appearance. The reason is that if an irregular surface is formed, light scattering occurs and the coated substrate will no longer function as an effect pigment.

In addition, the first coating should adhere strongly to the substrate or else the coating will become separated during processing, resulting in considerable breakage and loss of luster. Particles which do not become attached to the substrate during preparation of the coatings on the substrate or which are the result of separation cause light scattering and impart opacity to the pigment. When there are too many of such small particles, the pearlescent appearance can be reduced or lost.

U.S. Pat. No. 5,171,363 teaches a multilayer structure comprising alternate layers of a material having a low refractive index of 1.35 to 1.65 and a material having a high refractive index of 1.7 to 2.4. One example is silicon dioxide (refractive index of 1.5) and titanium dioxide (refractive index of 2.7). The layers are formed by vacuum coating, electron beam, or sputtering. The resulting optically variable flake is used to make optically variable ink.

Japanese Patent Application 7-246366 published Sep. 26, 1995 teaches that alternating layers of SiO₂ and TiO₂ may be sputtered or vaporized to form a pearlescent material for paint. The layers are applied to a substrate such as glass.

U.S. Pat. No. 4,879,140 teaches the use of plasma chemical vapor deposition to deposit multilayer films comprising alternating layers of SiO₂ and Si to a total of 30 layers for use as an interference filter. The patent also teaches deposition of alternating layers of SiO₂ and TiO₂ to a total of 31 layers having a total thickness of approximately 2 microns.

A need exists in the cosmetic and personal care markets for a pearlescent pigment having new effects.

### SUMMARY OF THE INVENTION

The present invention provides a cosmetic comprising a multilayer structure having a chemical or physical vapor deposited layer of a first material and a chemical or physical vapor deposited layer of a second material.

Preferably, the present invention uses materials exempt from certification according to present and future regulations of 21 CFR Part 73, Subpart A, B & C (Apr. 1, 2002 edition) in the multilayer structure.

### DETAILED DESCRIPTION OF THE INVENTION

The invention relates to the use of materials exempt from certification according to present and future regulations of 21 CFR Part 73 Subpart A, B & C (Apr. 1, 2002 edition) in multilayer laminar form for the use in cosmetics, drugs and foods. The resulting compositions are also part of this invention. To date, there are no multilayer laminar structured materials comprising exclusively of color additive mixtures exempt from certification according to present and future regulations of 21 CFR Part 73. Technological and economical circumstances heretofore have recently advanced to allow for the viability of such compositions. Such materials may be referred to cosmetic (C) drug and cosmetic (D&C) and/or food, drug & cosmetic materials (FD&C).

The phrase “non-optically active layer” as used herein means an internal or external layer associated with the optical package that provides non-limiting attributes other than optical effects such as adhesion promotion, mechanical integrity and control of interfacial contraction or expansion.

The phrase “US FDA acceptable material” as used herein means an additive exempt from US Food and Drug Administration certification under 21 CFR (Apr. 1, 2002 edition) and includes food grade materials in contact with drugs and food.

Metal oxide and hydroxide herein means all oxides and hydroxides of a specific metal, especially those exempt from US Food and Drug Administration certification under 21 CFR.

Color materials exempt from US Food and Drug Administration certification under 21 CFR Part 73 Subpart A—Foods (Apr. 1, 2002 edition) include diluents in color additive mixtures for food use exempt from certification, annatto extract, astaxanthin, dehydrated beets (beet powder), ultramarine blue, canthaxanthin, caramel, β-Apo-8′-carotenal, β-Carotene, cochineal extract; carmine, toasted partially defatted cooked cottonseed flour, ferrous gluconate, ferrous lactate, grape color extract, grape skin extract (enocianina), haematococcus algae meal, synthetic iron oxide, fruit juice, vegetable juice, dried algae meal, tagetes (Aztec marigold) meal and extract, carrot oil, corn endosperm oil, paprika, paprika oleoresin, phaffia yeast, riboflavin, saffron, titanium dioxide, turmeric, and turmeric oleoresin.

Color materials exempt from US Food and Drug Administration certification under 21 CFR Part 73 Subpart B—Drugs (Apr. 1, 2002 edition) include diluents in color additive mixtures for drug use exempt from certification, alumina (dried aluminum hydroxide), chromium-cobalt-aluminum oxide, ferric ammonium citrate, annatto extract, calcium carbonate, canthaxanthin, caramel, β-Carotene, cochineal extract; carmine, potassium sodium copper chlorophyllin (chlorophyllin-copper complex), dihydroxyacetone, bismuth oxychloride, synthetic iron oxide, ferric ammonium ferrocyanide, ferric ferrocyanide, chromium hydroxide green, chromium oxide greens, guanine, pyrogallol, pyrophyllite, logwood extract, mica, talc, titanium dioxide, aluminum powder, bronze powder, copper powder, and zinc oxide.

Color materials exempt from US Food and Drug Administration certification under 21 CFR Part 73 Subpart C—Cosmetics (Apr. 1, 2002 edition) include annatto, caramel, carmine, β-Carotene, bismuth citrate, disodium EDTA-copper, potassium sodium copper chlorophyllin (chlorophyllin-copper complex), dihydroxyacetone, bismuth oxychloride, guaiazulene, henna, iron oxides, ferric ammonium ferrocyanide, ferric ferrocyanide, chromium hydroxide green, chromium oxide greens, guanine, lead acetate, pyrophyllite, mica, silver, titanium dioxide, aluminum powder, bronze powder, copper powder, ultramarines, manganese violet, zinc oxide, and luminescent zinc sulfide. Preferred materials include titanium dioxide and zinc oxide.

In addition to the above mentioned materials, it is understood that any of the layers of the optical package may be made from any natural or synthetic polymer approved for ingestion including biodegradable materials.

The CRC Handbook of Chemistry and Physics, 63^{rd} edition reports refractive indices for some of these color materials as follows.

| Material | Refractive Index |
|---|---|
| TiO2 - anatase | 2.55 |
| TiO2 - rutile | 2.90 |
| Fe2O3 - hematite | 3.01 |
| ZnO | 2.03 |
| ZnS | 2.38 |
| BiOCl | 2.15 |

The multilayer laminar structure is formed as follows. Typically a polymeric support layer is used. Preferred polymers include poly(ethylene terephthalate); however, other metallic, ceramic or polymeric support layers are known in the art. The multilayer laminar structures contemplated in this invention may be produced from a variety of techniques including, however not limited to, chemical vapor deposition (CVD), and physical vapor deposition (PVD). The terms CVD and PVD are understood to be general terms encompassing microwave enhanced, plasma enhanced and radio frequency enhanced CVD as well as combinations thereof and evaporative deposition, reactive evaporative deposition, D.C. & R.F. sputtering, magnetron sputtering, electron beam, arc deposition respectively, and the like. A good reference source for some of these techniques can be found in Thin Materials, John L. Vossen and Warner Kern, eds., Academic Press, Inc., New York, N.Y. (1978) and US, incorporated herein as reference. U.S. Pat. No. 4,879,140 incorporated herein by reference discloses a useful plasma chemical vapor deposition process for making the present pearlescent pigment.

Preferably, the thickness of each layer ranges from about 5 nanometers (nm) to about 500 nm.

A significant advantage of the manufacturing methods involving CVD & PVD processes is their readily lending themselves to modification for clean room operations thus ensuring the cleanliness and purity requirements for highly regulated uses such as food, drug or cosmetics. For example, many target materials used in PVD can be obtained at extremely high purities. Additionally, CVD precursors, such as iron pentacarbonyl, may be utilized to obtain extremely high purity end products. U.S. Pat. No. 6,186,090 on for “Apparatus for the Simultaneous Deposition By Physical Vapor Deposition and Chemical Vapor Deposition and Method Thereof”, the disclosure of which is incorporated herein by reference, teaches an apparatus for simultaneous multilayer laminar film deposition through differing processes. PVD is useful for forming metal layers while plasma enhanced CVD is useful for forming metal oxide layers.

After the appropriate multilayer laminar structure has been deposited on the carrier web, it is necessary to remove the structure from the carrier web in the form of particulates so they may be used in conventional pigment and/or effect material applications. For a preview of well established techniques for removing multilayer film structures from a carrier web in the form of particulate while maintaining multilayer laminar character and particle size control, see U.S. Pat. Nos. 4,321,087; 5,059,454 and 6,398,999 for their teachings, all of which are hereby incorporated by reference.

It may be necessary for improved performance to provide the particulate with a surface treatment. Treatments are often used to improve stability, tactile and mechanical and optical properties of the particulate.

Analytical Color Measurement Methods:

Desirable product attributes most often focus on the luster and color which is evaluated using drawdowns on a hiding chart (Form 2-6 Opacity Charts of the Leneta Company both visually and instrumentally. A drawdown on the black portion of the card displays the reflection color while the white portion displays the transmission color at non-specular angles.

A typical drawdown preparation involves incorporating 3-12% multilayer laminar particulate in a nitrocellulose lacquer, with the concentration dependent on the particle size distribution. For example, a 3% drawdown would likely be used for an average particle size of 20 μm while a 12% drawdown might be used for an average particle size of 100 μm. The particulate-nitrocellulose suspension is applied to the drawdown card using a Bird film application bar with a wet film thickness of 3 mil. The drawdown is further characterized using a goniospectrophotomerer (CMS-1500 from Hunter). The reflectivity v. wavelength curve is obtained at various viewing angles. The color travel for the multilayer laminar particulate is described using the CIELab L*a*b* system. The data is recorded both numerically and graphically.

Utility: In the cosmetic area, the present pigments may be used in the eye area and in all external and rinse-off applications. Thus, they can be used in hair sprays, face powder, leg-makeup, insect repellent lotion, mascara cake/cream, nail enamel, nail enamel remover, perfume lotion, and shampoos of all types (gel or liquid). In addition, they can be used in shaving cream (concentrate for aerosol, brushless, lathering), skin glosser stick, skin makeup, hair groom, eye shadow (liquid, pomade, powder, stick, pressed or cream), eye liner, cologne stick, cologne, cologne emollient, bubble bath, body lotion (moisturizing, cleansing, analgesic, astringent), after shave lotion, after bath milk and sunscreen lotion.

### INVENTIVE EXAMPLE 1

Titanium dioxide is exempt from FDA certification under 21 CFR §73.2575 (Apr. 1, 2002 edition) and copper powder is exempt from FDA certification under 21 CFR §73.2647 (Apr. 1, 2002 edition). A multilayer structure comprising Cu/TiO₂/Cu/TiO₂/Cu is made using PVD for formation of the Cu layers and plasma enhanced CVD for formation of the TiO₂ layers.

A cosmetic is made using the resulting pearlescent pigment.

### INVENTIVE EXAMPLE 2

Iron oxide is exempt from FDA certification under 21 CFR §73.2250 (Apr. 1, 2002 edition) and copper powder is exempt from FDA certification under 21 CFR §73.2647 (Apr. 1, 2002 edition). A multilayer structure comprising Cu/Fe₂O₃/Cu/Fe₂O₃/Cu is made using PVD for formation of the Cu layers and plasma enhanced CVD for formation of the Fe₂0₃ layers.

A cosmetic is made using the resulting pearlescent pigment.

### INVENTIVE EXAMPLE 3

Iron oxide is exempt from FDA certification under 21 CFR §73.2250 (Apr. 1, 2002 edition) and titanium dioxide is exempt from FDA certification under 21 CFR §73.2575 (Apr. 1, 2002 edition). A stacked structure comprising TiO₂/Fe₂O₃/TiO₂/Fe₂O₃/TiO₂ is made using plasma enhanced CVD for layer formation.

A cosmetic is made using the resulting pearlescent pigment.

### INVENTIVE EXAMPLE 4

Titanium dioxide is exempt from FDA certification under 21 CFR §73.2575 (Apr. 1, 2002 edition) and zinc oxide is exempt from FDA certification under 21 CFR §73.2991 (Apr. 1, 2002 edition). A stacked structure comprising Fe₂O₃/ZnO/Fe₂O₃/ZnO/Fe₂O₃ is made.

The resulting pearlescent pigment is incorporated into a nail enamel. 10 g of HIO-GEM is mixed with 82 g of suspending lacquer SLF-2, 4 g lacquer 127 P, and 4 g ethyl acetate. The suspending lacquer SLF-2, is a generic nail enamel consisting of butyl acetate, toluene, nitrocellulose, tosylamide/formaldehyde resin, isopropyl alcohol, dibutyl phthalate, ethyl acetate, camphor, n-butyl alcohol and silica.

### INVENTIVE EXAMPLE 5

A stacked structure comprising Si/SiO₂/Si/Si0₂/Si is made using PVD to form the Si layers and plasma enhanced CVD to form the Si0₂ layers.

### INVENTIVE EXAMPLE 6

A pearlescent pigment comprising Si₃N₄/Si0₂/Si₃N₄/Si0₂/Si₃N₄ is made using plasma enhanced CVD to form the layers.

### INVENTIVE EXAMPLE 7

A pearlescent pigment comprising A1/Sio₂/A1/Si0₂/A1 is made using PVD to form the A1 layers and plasma enhanced CVD to form the Si0₂ layers.

### INVENTIVE EXAMPLE 8

A pearlescent pigment comprising Cu/Si0₂/Cu/Si0₂/Cu is made using PVD to form the Cu layers and plasma enhanced CVD to form the Si0₂ layers.

Various changes and modifications can be made in the process and products of the invention without departing from the spirit and scope thereof. The various embodiments disclosed herein were for the purpose of illustration only and were not intended to limit the invention.

What we claim is:

1. Cosmetic comprising pearlescent pigment wherein said pearlescent pigment comprises:
(a) a CVD or PVD deposited layer of a first material; and
(b) a CVD or PVD deposited layer of a second material.

2. The cosmetic of claim 1 wherein said layer (a) is a US FDA acceptable material.

3. The cosmetic of claim 1 wherein said layer (b) is a US FDA acceptable material.

4. The cosmetic of claim 1 additionally comprises (c) a CVD or PVD deposited layer of said first material.

5. The cosmetic of claim 4 wherein said first material is selected from the group consisting of copper, titanium dioxide, and iron oxide.

6. The cosmetic of claim 4 wherein said second material is selected from the group consisting of titanium dioxide, iron oxide, and zinc oxide.

7. The cosmetic of claim 4 wherein said layer (a) is copper, said layer (b) is titanium dioxide, and said layer (c) is copper.

8. The cosmetic of claim 4 wherein said layer (a) is copper, said layer (b) is iron oxide, and said layer (c) is copper.

9. The cosmetic of claim 4 wherein said layer (a) is titanium dioxide, said layer (b) is iron oxide, and said layer (c) is titanium dioxide.

10. The cosmetic of claim 4 wherein said layer (a) is iron oxide, said layer (b) is zinc oxide, and said layer (c) is iron oxide.

11. Nail enamel comprising lacquer and said cosmetic of claim 1.
